(19) United States
(12) Patent Application Publication
(10) Pub. No.: US 2009/0105792 A1
(43) Pub. Date: Apr. 23, 2009

(54) Method and Devices for Treating Damaged Articular Cartilage

(76) Inventor: Theodore R. Kucklick, Los Gatos, CA (US)

Correspondence Address: CROCKETT & CROCKETT, P.C., 26020 ACERO, SUITE 200, MISSION VIEJO, CA, 92691, US

(21) Appl. No.: 11/875,121

(22) Filed: Oct. 19, 2007

(51) Int. Cl. A61N 5/06; A61B 18/18

(52) U.S. Cl. 607/89; 606/45

(57) ABSTRACT

The methods and devices described below provide for minimally invasive method of performing micro-fracture therapy for the treatment of osteoarthritis. The method entails use of a laser to ablate numerous holes into the bone and cartilage of a joint which is afflicted with osteoarthritis. Laser systems which are adapted to file several beams simultaneously to create several micro-fracture bores simultaneously to speed the creation of numerous micro-fracture bores in the cartilage and underlying bone.

### FIELD OF THE INVENTIONS

The inventions described below relate the treatment of osteoarthritis and other damage to articular cartilage.

### BACKGROUND OF THE INVENTIONS

Damage to the cartilage may result from traumatic injury or disease. Sports injuries are one typical cause of damage, and disease such as osteoarthritis is another typical cause. Osteoarthritis is the most common form of arthritis, and refers to the degradation of articular cartilage (cartilage in the joints) and condyle surfaces of bones (surface that abut other bones in a joint). There is no cure for osteoarthritis, and the disease can only be treated by ameliorating its symptoms and effects. Painkillers provide relief for many patients with moderate osteoarthritis. Treatments for more advanced cases of osteoarthritis include lavage and debridement (shaving the bearing surfaces of bones in a joint), fusing of the bones in the affected joint, and joint replacement. As an intermediate treatment, for cases in which the cartilage is moderately degraded, physicians may cut clean holes in the cartilage and then punch holes underlying subchondral bone, to cause bleeding in the bone. After puncture, some bone marrow seeps out of the holes with the blood, and this releases stem cells into the defect in the cartilage. The bone marrow blood coagulated into what is known as a super-clot. After healing, the cartilage includes fibro-cartilage, which is not as strong and healthy as normal hyaline cartilage, but is a good improvement over the diseased cartilage which it replaces. This does not provide a permanent cure, but is viewed as a valuable procedure because it results in acceptable joint function for several years, thus delaying the need for more aggressive surgeries. When used to treat traumatic injury, the cartilage can support rigorous athletic activity for years.

### SUMMARY

The methods and devices described below provide for minimally invasive method of performing micro-fracture therapy for the treatment of osteoarthritis. The method entails use of a laser to drill or ablate numerous holes into the bone and cartilage of a joint which is afflicted with osteoarthritis. Laser systems which are adapted to file several beams simultaneously to create several micro-fracture bores simultaneously to speed the creation of numerous micro-fracture bores in the cartilage and underlying bone.

### BRIEF DESCRIPTION OF THE DRAWINGS

FIG. 1 illustrates a method of performing micro-fracture using a penetrating laser.

FIGS. 2 and 3 illustrate laser probes which facilitate performance of the micro-fracture method.

FIGS. 4 and 5 illustrate patches adapted for placement over the micro-fracture field to aid in the regeneration of articular cartilage.

### DETAILED DESCRIPTION OF THE INVENTIONS

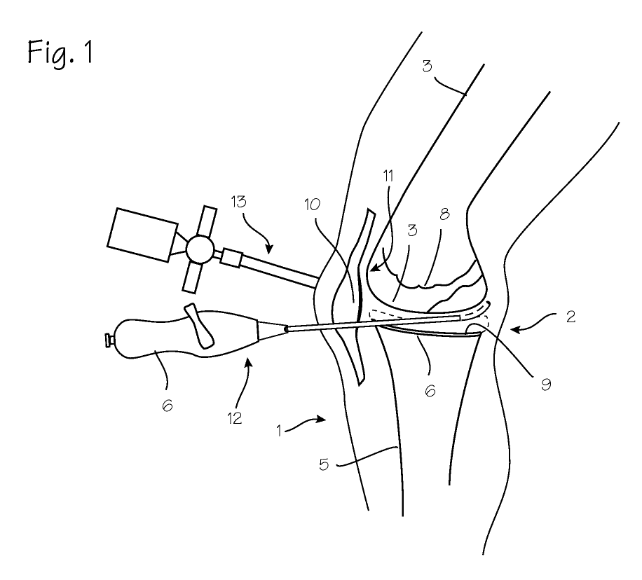

FIG. 1 illustrates a method of performing micro-fracture using a penetrating laser. The anatomy shown in FIG. 1 includes the patient's leg 1, including the knee joint 2 including the femur 3 and the femoral condyle 4, the tibia 5 the condyle 6 of the tibia, and the knee cap 7. A sheath of articular cartilage 8 covers the femoral condyle, and a sheath of articular cartilage 9 covers the tibial condyle, and facies anterior patellae covers the anterior surface of knee cap. The meniscus 10 (the lateral and medial meniscus) supports the knee joint. The articular cartilage and underlying bone (referred to as the subchondral plate) of the condyles are subject to the damage of osteoarthritis. Damage may appear in the trochlear groove 11 the anterior surface of the patella, which are fairly easy to reach in an arthroscopic procedure, but can also appear on the posterior condyle 4p and other hard-to-reach areas.

To perform the laser micro-fracture procedure, a surgeon inserts a side-firing tip of an laser probe 12 into a surgical work space proximate the target bone of the subchondral plate (which underlies the position of the cartilage lesion), aligns the laser emitting ports toward the target bone tissue, and energizes the laser probe with sufficient laser power to bore holes through the bone tissue into the marrow within the bone. In an exemplary procedure, the surgeon will use the laser to create laser bore holes over a 2 cm by 2 cm area bone tissue, manipulating the tip of the probe prior and energizing the laser as necessary to create a number of bore holes distributed uniformly over the target area of the bone. Preferably, the surgery is accomplished arthroscopically, with an arthroscope 13 inserted into the arthroscopic workspace (the joint capsule) so that the surgeon can view the surgical field. The laser probe can be provided with a steerable distal tip, and steering actuator on the proximal handle, to provide some capacity to steer around joint structures and reach difficult access points through access ports installed on the patient's joints according to standard protocols. Preferably, the laser source used with the probe may be an Erbium/YAG, Erbium, Excimer, femtosecond laser, operated at power levels sufficient to ablate bore-holes through the condyle bone tissue.

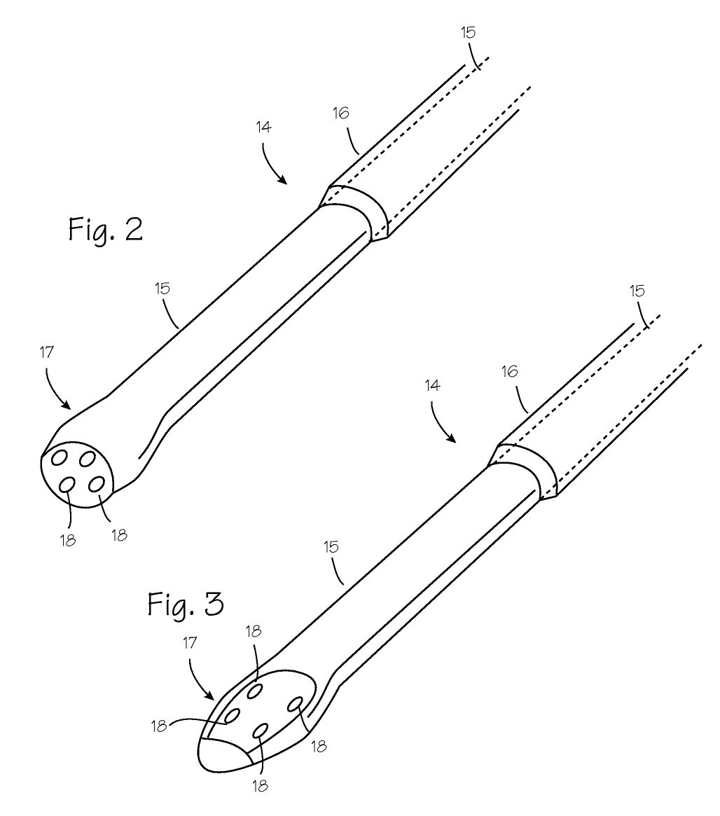

FIGS. 2 and 3 illustrate laser probes which facilitate performance of the micro-fracture method. The laser probes 14 comprise a laser catheter 15 slidably disposed within a cannula 16, such that the laser catheter may be translated within the surgical space while the cannula is held fixed relative to the surgical space. The laser head 17 in each probe includes multiple outlets 18 to direct laser energy into the bone tissue. In FIG. 2, the apertures are arranged in an end-firing arrangement, while in FIG. 3, the apertures are arranged in a side firing relationship. The end-firing embodiment is suited for directing laser energy to the femoral condyle and the patella in the knee joint. The side-firing embodiment is suited for directing laser energy to the femoral head in the hip joint, or the humeral head in the shoulder, and can be retracted or advanced relative to the subchondral bone surface to create a track or field of uniformly dispersed perforation in the chondral surface. The probes may be used to treat osteoporosis and traumatic cartilage damage in various joints of the body, including the knees, hips, elbows, and shoulders.

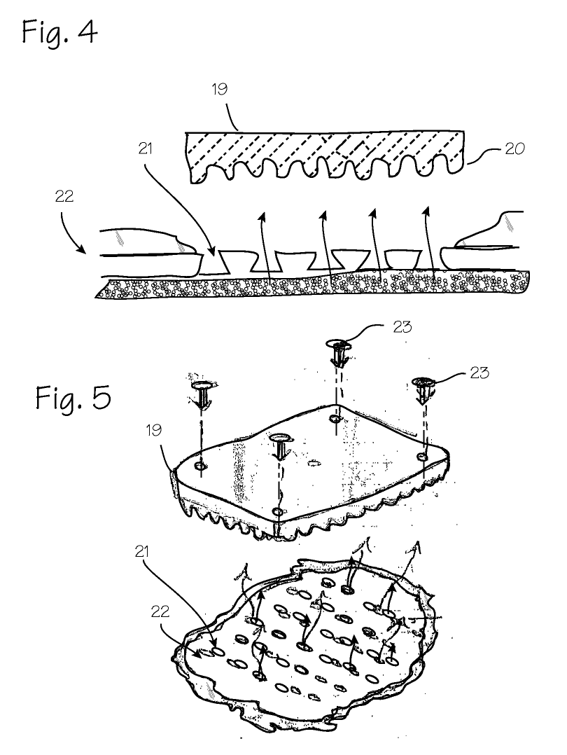

The microfracture method may be augmented by implantation of a natural or synthetic material to act as a stimulant for new cartilage growth or as a scaffold for new cartilage growth. Suitable scaffolding material may include metals, plastics, hydrogels. Plastic scaffolds may comprise UHMWPW (ultra high molecular weight polyethylene) or PEEK (poly ether-ether ketone). Tissue engineered constructs consisting of a hydrogel seeded with chondrocytes and growth factors which favorably binds and integrates with chondral tissue by means of growth factors released from the bone marrow. Hydrogels comprising hyaluronan may also be used to promote cartilage regrowth. Materials such as a non-absorbable polyvinylidene fluoride (PVDF) or absorbable polyglactin or polylactic acid (PLA) may be used. Metal scaffolds may comprise cobalt-chrome alloy, stainless steel or titanium. Each of these materials may be provided in the form of small sheets or patches that may be applied to the subchondral bone surface before or after the microfracture procedure has been used to create numerous perforations in the subchondral bone. The tissue implant side of the sheet may have a textured surface, and may be formed with pins or protrusions that fit into the perforations produced by the laser microfracture device to promote optimal tissue ingrowth and integration. The patch may be held in place with resorbable pins, penetrating the patch and engaging a hole created by the laser. Where the patch is made of deformable plastic material, it may also be pressed into the holes to fix it to the subchondral plate. FIGS. 4 and 5 illustrate patches adapted for placement over the micro-fracture field to aid in the regeneration of articular cartilage. FIG. 4 illustrate a patch 19 with a number of surface features 20, analogous to tenons, sized and dimensioned to fit into the holes 21, which act as mortises in the subchondral plate 22. FIG. 5 illustrates a patch 19 which is secured to the subchondral plate with several pins 23 adapted to securely fit into the holes 21 created in the subchondral plate 22. The stem cells in the super-clot may also promote adhesion and integration of implants with ceramic coatings such as hydroxyapatite.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. A method of treating damage to articular cartilage of a joint comprising:
excising damaged articular cartilage from the joint to expose a portion of the subchondral plate;
operating a laser to create a plurality of holes in the subchondral bone to cause bleeding from the plate;
allowing the bone and articular cartilage to heal.

2. A method of promoting regeneration of articular cartilage of a joint, comprising:
providing a laser probe having a plurality of laser emitting apertures;
inserting said laser probe into an arthroscopic work space created around the joint;
operating the laser probe to a plurality of holes in a subchondral plate of an area of said joint to be treated;
translating the laser probe within the operating space and repeating the operation of the laser prove to form additional holes to create a field of holes in the subchondral plate;
allowing said area to regenerate cartilage.

3. A method of promoting regeneration of articular cartilage of a joint, comprising:
forming a plurality of holes in a subchondral plate of an area of said joint to be treated;
covering the area to be treated, including the plurality of said holes, with a patch;
fixing the patch over said area; and
allowing said area to regenerate cartilage;
wherein the step of forming holes is accomplished by laser ablation of the subchondral plate.

4. A method of claim 2 further comprising the steps of:
providing the patch with a plurality of surface features adapted to engage one or more holes in the subchondral plate, and placing said patch over the area to be treated so that said surface features engage the holes.

5. A method of claim 3 further comprising the steps of:
providing the patch with a plurality of surface features adapted to engage one or more holes in the subchondral plate, and placing said patch over the area to be treated so that said surface features engage the holes.

6. A method of claims 1 further comprising the steps of:
providing the patch with a plurality of surface features adapted to engage one or more holes in the subchondral plate, and placing said patch over the area to be treated so that said surface features engage the holes.
